(19) United States
(12) Patent Application Publication
(10) Pub. No.: US 2012/0197263 A1
(43) Pub. Date: Aug. 2, 2012

(54) Instrument Set for Screwing an Implant Into an Intervertebral Disc Space

(75) Inventors: Franz Copf, Stuttgart (DE); Marcus Mohr, Gerlingen (DE); Lino Taddia, Stockach (DE); Erik Fischer, Stuttgart (DE); Tomislav Anic, Stuttgart (DE); Gerhard Pross, Weil im Schonbuch (DE)

(73) Assignee: Spontech Spine Intelligence Group AG, Stuttgart (DE)

(21) Appl. No.: 13/220,499

(22) Filed: Aug. 29, 2011

(30) Foreign Application Priority Data
Aug. 30, 2010 (DE) 10 2010 035 832.0

(51) Int. Cl. A61B 17/56

(52) U.S. Cl. 606/104

(57) ABSTRACT

An instrument set for screwing an implant into an intervertebral disc space comprises a guide sleeve and a screwing-in instrument. The latter has a shank having a distal end at which the implant can be fixed in a rotationally rigid manner. At the proximal end a rotary handle is fastened in a rotationally rigid manner. The screwing-in instrument further has a residual-advance indicator, which is configured to quantitatively indicate to the surgeon, during the screwing-in procedure, how far the implant is distant in the axial direction from an axial specified position that is fixed relative to the location at which the guide sleeve is supported on the vertebrae.

### CROSS-REFERENCE TO RELATED APPLICATIONS

This application claims priority of German patent application DE 10 2010 035 832.0 filed Aug. 30, 2010. The full disclosure of this earlier application is incorporated herein by reference.

### BACKGROUND OF THE INVENTION

1. Field of the Invention

The invention relates to an instrument set for screwing an implant that is provided with an external thread into an intervertebral disc space.

2. Description of the Prior Art

In the case of degenerated vertebral column segments, a possible treatment consists in fully or partially removing the affected vertebral disc and inserting an implant in the intervertebral disc space. Frequently, the implant is provided with an external thread, and can thereby be screwed into the prepared intervertebral disc space. The implant holds the adjacent vertebrae apart and enables the intervertebral disc space gradually to become ossified, which ultimately results in fusion of the adjacent vertebrae.

An example of such an implant is described in WO 2005/082292 A1. The known implant has a rounded head having a conical threaded portion, a central portion having large-area windows, and a base portion that has fastening means for a screwing-in instrument.

Before the implant can be screwed in, it is generally necessary to distract the intervertebral disc space. For this, it is possible to use, for example, a system composed of a plurality of guide sleeves and distractors that are matched to one another, as described in as it is described in U.S. Ser. No. 13/215,535.

After distraction has been completed, two spacer tongues protruding at the distal ends of the guide sleeves project into the intervertebral disc space, and maintain the distraction. After the last distractor has been removed, a screwing-in instrument can be introduced through the last guide sleeve to be used, the implant being attached to the distal end of the screwing-in instrument.

The screwing-in of the implant is a critical step insofar as the axial position of the implant in the intervertebral disc space is finally fixed as a result. If the implant does not assume the desired axial specified position, this can cause renewed discomfort for the patient and result, for example, in pathological changes in adjacent vertebral column segments. Moreover, particularly when the implant is screwed too far into the intervertebral disc space, there is the risk of nerves, blood vessels or other sensitive tissue being damaged by the implant.

This is complicated by the fact that the surgeon, during the screwing-in procedure, generally cannot see the implant from outside. Although continuous monitoring of the entire screwing-in procedure is possible with the aid of a fluoroscope or other imaging method, it is problematic for reasons of radiation exposure. For this reason, it would be optimal if the surgeon were also able to screw in the implant as precisely as possible without the aid of imaging methods, i.e. using only the instrument set available to the surgeon, until the implant has reached its specified position. Monitoring by imaging methods is then preferably performed only in a subsidiary manner, for instance in order to effect a final check at the end of the screwing-in procedure.

The screwing-in instruments known hitherto usually have, at their distal end, a special receiver, to which the implant can be fastened in a rotationally rigid and axially secured manner. Following completion of the screwing-in procedure, this fastening is released, such that the screwing-in instrument can be withdrawn from the guide sleeve. Some screwing-in instruments in this case are provided with adjustable stops, which stop against the guide sleeve and prevent further penetration when the implant has reached an axial specified position determined by the surgeon.

US 2006/0116688 A1 describes, for example, a stop that can be fastened in a releasable manner, in various axial positions, to the shank of a screwing-in instrument. The stop is provided with a viewing window, through which can be seen numbers applied to the shank. The numbers in this case indicate how far the distal end of the shank projects out of the guide sleeve when the stop stops against the guide sleeve.

In the case of the known stops, however, it is disadvantageous that, although the implant is prevented from penetrating too deeply into the intervertebral disc space, the surgeon can nevertheless only ascertain very imprecisely during the screwing-in procedure how far distant the implant still is from its specified position. This is because, generally, the surgeon will set the implant so as merely to avoid endangering sensitive tissue. However, the desired specified position is generally at a somewhat lesser distance from the surgeon, and the surgeon cannot read off from the instrument when this specified position has been reached. Clearly, the surgeon could also fix the stop axially such that, when the screwing-in instrument is in the end position, the implant has reached its specified position. Then, however, it would no longer be possible for the surgeon to make smaller adjustments also towards the distal end without releasing the stop and thereby running the risk of inadvertently penetrating too deeply into the intervertebral disc space.

### SUMMARY OF THE INVENTION

It is therefore the object of the invention to specify an instrument set for screwing an implant, provided with an external thread, into an intervertebral disc space, which instrument set enables safer screwing-in of the implant as far as the predefined specified position.

This object is achieved, according to the invention, by an instrument set having a guide sleeve, which has a distal end and is supported, during the screwing-in procedure, on vertebrae delimiting the intervertebral disc space. The guide sleeve, further, has a proximal end, which faces towards the surgeon during the screwing-in procedure. The instrument set further includes a screwing-in instrument, which has a shank that can be introduced into the guide sleeve. The shank has a distal end, to which the implant can be fixed in a rotationally rigid manner, and has a proximal end, to which a rotary handle is fastened in a rotationally rigid manner and which projects over the proximal end of the guide sleeve during the screwing-in procedure. According to the invention, the screwing-in instrument has a residual-advance indicator, which quantitatively indicates to the surgeon, during the screwing-in procedure, how far the implant is distant in the axial direction from an axial specified position that is fixed relative to the location at which the guide sleeve is supported on the vertebrae.

The residual-advance indicator according to the invention enables the surgeon carefully to approach the axial specified position of the implant and to position the implant there, even without the assistance of imaging methods. Thus, instead of relying only on a stop, which becomes effective only when a critical axial depth position has been reached, the surgeon can identify from the residual-advance indicator how far distant from the specified position he is, and can then discontinue the screwing-in procedure when the specified position has been reached. The hitherto comparatively imprecise working in the proximity of a stop position is replaced, according to the invention, by a targeted and careful approaching of the specified position, which is rendered possible only by the residual-advance indicator according to the invention.

Quantitative indication in this context means, in particular, that the surgeon can identify, from lines of a scale or from other uniformly graduated marks, how many graduation units remain to be covered until the specified position is reached. Clearly, the markings can also be supplemented or replaced by numerals or other symbols, for instance a descending sequence of capital letters (E, D, C, B, A). Non-uniformly graduated lines or marks can also be used for the quantitative indication of the residual advance, provided that it is ensured that the surgeon can reliably estimate the advance that remains until the specified position is reached. Thus, for example, just a graduation that becomes increasingly narrower can make it clear to the surgeon that the implant is getting ever closer to its specified position.

The residual-advance indicator can be realized in such a way that it also quantitatively indicates exceeding of the specified position in the axial direction. In this way, it becomes possible for the surgeon to effect a fine adjustment of the axial position of the implant and, in so doing, always to see on the residual-advance indicator how far the implant is distant, in both directions, from its specified position.

The residual-advance indicator can comprise a scale, which is applied to the shank or to a component fixed axially thereto, and which acts together with the guide sleeve, this being, in particular, with the proximal end thereof. For this purpose, the guide sleeve can be provided, for example, with a window, through which the scale on the shank can be seen, and applied to the edge of which there is a marking that points to the scale and thereby indicates the residual advance. For this purpose, the scale can be inscribed, for example, with numbers that indicate, in a predefined unit of length, the axial distance between the implant and the specified position.

It is simplest, however, if the proximal end of the guide sleeve directly or a (e.g. pointer-type) marking applied thereto points to the scale and thereby indicates the residual advance when the screwing-in instrument has been introduced into the guide sleeve.

The screwing-in instrument can have an indicator sleeve, which is guided so as to be axially movable relative to the shank and which, during the introduction of the screwing-in instrument into the guide sleeve, or at least in the course of the screwing-in procedure, stops against the proximal end of the guide sleeve, and is provided with a viewing window, through which the scale can be seen. In this way, the scale is shifted, as it were, in the axial direction to the proximal end, i.e. towards the surgeon. This is advantageous insofar as the surgeon can thereby read off the scale more easily than if it is located in the region of the guide sleeve.

For this purpose, the edge of the viewing window can be provided, for example, with a pointer-type marking directed towards the scale.

Further, the screwing-in instrument can have an elastic element, which biases the indicator sleeve in the axial direction in such a way that, during the screwing-in procedure, the indicator sleeve is displaced axially relative to the shank, against the action of the elastic element. The bias thereby ensures that the indicator sleeve, after stopping for the first time against the guide sleeve, remains in this position and is not unintentionally removed again from the guide sleeve.

The scale can have a first scale portion, which acts together with the marking on the guide sleeve, and a second scale portion, which acts together with the marking on the indicator sleeve.

In particular, the two scale portions can be matched to one another in such a way that, when the indicator sleeve stops against the proximal end of the guide sleeve during the screwing-in procedure, it is solely the second scale portion that quantitatively indicates the residual advance and, possibly, exceeding of the specified position in the axial direction. Through such a change in the residual-advance indicator between the different scale portions, the surgeon is additionally made aware that the implant is now in immediate proximity of the specified position, and that the further screwing-in procedure should be performed with particular care.

The residual advance can be less than −5 mm, preferably less than −2.5 mm, when, during the screwing-in procedure, the indicator sleeve stops against the proximal end of the guide sleeve and, as a result, the residual advance can be seen on the second scale portion.

The guide sleeve can have, at a distal end, two spacer tongues that engage in the intervertebral disc space when the guide sleeve is supported on the vertebrae during the screwing-in procedure.

The shank of the screwing-in instrument can have a through-bore extending over the entire length of the shank. Further, the screwing-in instrument can have a fastening rod, which is guided in the through-bore and which, at its distal end, is provided with a fastening portion, by means of which the implant can be fixed to the fastening rod in the axial direction.

For this purpose, the fastening portion can have an external thread, which is arranged to act together with a complementary internal thread realized in a bore on the implant.

Further, the fastening rod can carry, at its proximal end, a locking handle, upon actuation of which the fastening rod rotates about its longitudinal axis.

The rotary handle assigned to the shank can be detachably fastened to the shank. It is thereby possible for one and the same rotary handle to be fastened to shanks of different dimensions.

For the purpose of fastening the rotary handle to the shank, the screwing-in instrument can have a latching mechanism, the latching of the rotary handle with the shank being releasable only through actuation of a spring-loaded latching button. This prevents the rotary handle from being unintentionally released from the shank and having to be refitted.

The fixing of the rotary handle to the shank in a rotationally rigid manner can be achieved by positive locking, such that the latching mechanism need fix the rotary handle solely in the axial direction relative to the shank. To achieve the positive locking, plane surfaces can be provided on the rotary handle and on the shank, which plane surfaces are aligned in such a way that the rotary handle can be pushed laterally onto the shank, and the rotary handle, when in the pushed-on state, is fixed on the shank in a rotationally rigid and positive manner.

The latching mechanism can then have, for example, a latching sleeve, which is screwed onto the shank and which, when in the latched state, is fixed in position axially and possibly also fixed in a rotationally rigid manner on the shank.

Further, the latching mechanism can be arranged such that latching occurs only when the latching mechanism has produced a predefined number of clearly audible click sounds (sound pressure, in particular, >20 dB). The click sounds then indicate to the surgeon or to an assistant that the latching has occurred and that the rotary handle has been securely fastened to the shank.

Subject-matter of the invention, further, is an implantation system comprising an implant that is provided with an external thread and arranged for screwing into an intervertebral disc space, and comprising an instrument set according to the invention. The implant has different dimensions in two orthogonal directions that run perpendicularly in relation to a longitudinal axis of the implant, this being in such a way that, depending on the angular orientation of the implant in the intervertebral disc space, different distances and/or angles between the vertebrae delimiting the intervertebral disc space are obtained. A fastening portion of the screwing-in instrument is arranged in such a way that the implant can be fixed to the screwing-in instrument in a rotationally rigid manner only in precisely two angular orientations, which differ from one another by 180°. Owing to this definite fixing of the implant to the screwing-in instrument, the orientation of the implant in the intervertebral disc space can be identified on the screwing-in instrument itself, without the need for imaging methods.

For this purpose, for example, the rotary handle on the screwing-in instrument can have a portion that, in a section perpendicular to the longitudinal axis of the shank, has at least substantially the shape of a rectangle, the long sides of the rectangle running parallel to the direction along which the dimensions of the implant fixed to the screwing-in instrument are greater.

The rotationally rigid fixing of the implant to the screwing-in instrument can be achieved, in particular, through positive locking. Further, an external thread on the fastening portion can be disposed axially in such a way that only in the two angular orientations can it engage in an internal thread on the implant.

The implantation system can additionally comprise a plurality of shanks, which differ from one another in the design of the fastening portions, and a plurality of implants of different sizes, which have fastening means by which they can each be fixed in a rotationally rigid manner to the fastening portion of one of the shanks. In this case, at least two implants that differ in size also have differently designed fastening means. Further, the system comprises a plurality of guide sleeves, the inside diameters of which are matched to the diameters of the implants and of the shanks.

The system can also comprise a plurality of fastening rods, which are matched to the different implants. Preferably, different screwing-in instruments can then be obtained by fitting together one of a plurality of different fastening rods, a shank matching the same, and otherwise identical components.

### BRIEF DESCRIPTION OF THE DRAWINGS

Further features and advantages of the invention will become apparent from the following description of preferred embodiments, with reference to the drawings, wherein:

FIG. 1 is a perspective view of a guide sleeve, which is part of the instrument set according to the invention;

FIG. 2 is a front view of the guide sleeve shown in FIG. 1;

FIG. 3 is a side view of the guide sleeve shown in FIG. 1;

FIG. 4 is a bottom view of the guide sleeve shown in FIG. 1;

FIG. 5 is a perspective view of a screwing-in instrument, which is likewise part of the instrument set according to the invention, together with an implant to be screwed in;

FIG. 6 is a perspective exploded view of an assembly of the screwing-in instrument shown in FIG. 5;

FIG. 7 is a perspective view of a part of the assembly shown in FIG. 6, in the assembled state;

FIG. 8 is a perspective exploded view of the screwing-in instrument shown in FIG. 5, but without handle unit;

FIG. 9 is a perspective view of the screwing-in instrument shown in FIG. 5, but with the handle unit drawn off laterally;

FIG. 10 shows an axial section through the screwing-in instrument shown in FIG. 5, with an implant fastened thereto;

FIG. 11 shows an axial section through the handle unit and the parts of the screwing-in instrument adjoining the same, before latching of a latching sleeve;

FIG. 12 shows a detail from FIG. 11, but after latching of the latching sleeve;

FIG. 13 shows a cross-section through the screwing-in instrument shown in FIG. 5, at the level of the latching sleeve;

FIG. 14 shows a detail, based on FIG. 12, for an alternative configuration of a latching mechanism;

FIG. 15 is a side view of the guide sleeve shown in FIG. 1, but introduced into an intervertebral disc space,

FIG. 16 is a front view of the guide sleeve according to FIG. 15 introduced into the intervertebral disc space;

FIG. 17 is a perspective view of the screwing-in instrument shown in FIG. 5 and of a plurality of different shanks and fastening rods;

FIG. 18 is a perspective view of the distal end of the screwing-in instrument with an implant fastened thereto;

FIG. 19 shows an axial section through the distal end shown in FIG. 18;

FIG. 20 is a front view of the screwing-in instrument introduced into the guide sleeve, before commencement of the screwing-in procedure;

FIG. 21 is a front view of the implant screwed into the intervertebral disc space, in its axial specified position, with guide sleeve cut open;

FIGS. 22a to 22e show details from FIG. 20, but in different axial positions of the screwing-in instrument.

### DESCRIPTION OF PREFERRED EMBODIMENTS

### I. Structure

The instrument set, according to the invention, for screwing an implant, provided with an external thread, into an intervertebral disc space comprises a plurality of guide sleeves, and comprises a screwing-in instrument, which, through exchange of a shank and a fastening rod, can be matched to the respectively selected implant. Described more fully in the following, firstly, is one of the guide sleeves; the other guide sleeves differ from it only in particular dimensions.

### a) Guide Sleeve

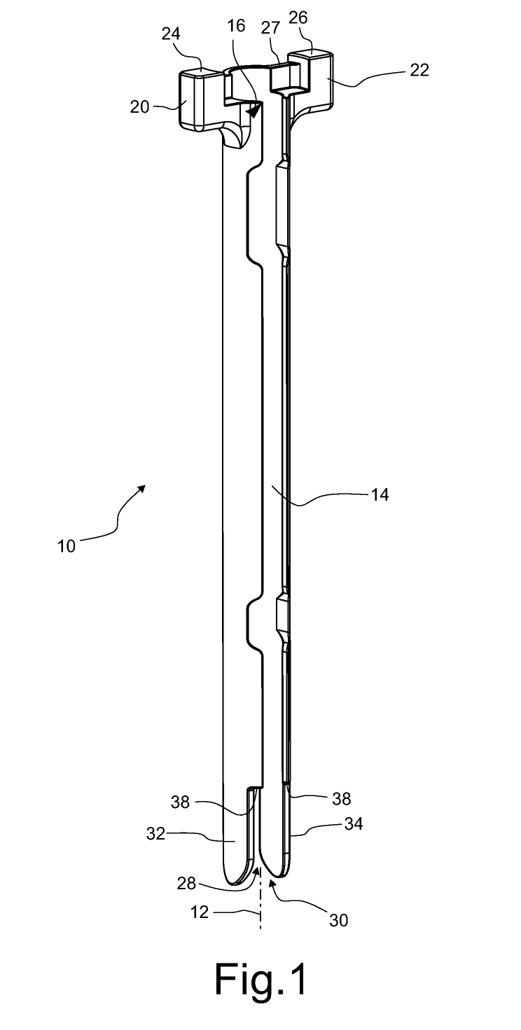

The structure of one of the guide sleeves is described more fully in the following with reference to FIGS. 1 to 4. These figures show the guide sleeve in a perspective view, a front view, a side view, and a bottom view, respectively.

The guide sleeve, which is denoted as a whole by 10, has a proximal end, which, in the figures, faces upwards, and which faces towards the surgeon when the guide sleeve 10 is being used during an intervertebral disc operation. The opposite end of the guide sleeve 10, which faces downwards in FIGS. 1 to 3, is termed the distal end. During the operation, the guide sleeve 10 is introduced with the distal end foremost, through a previously exposed access canal, into the patient.

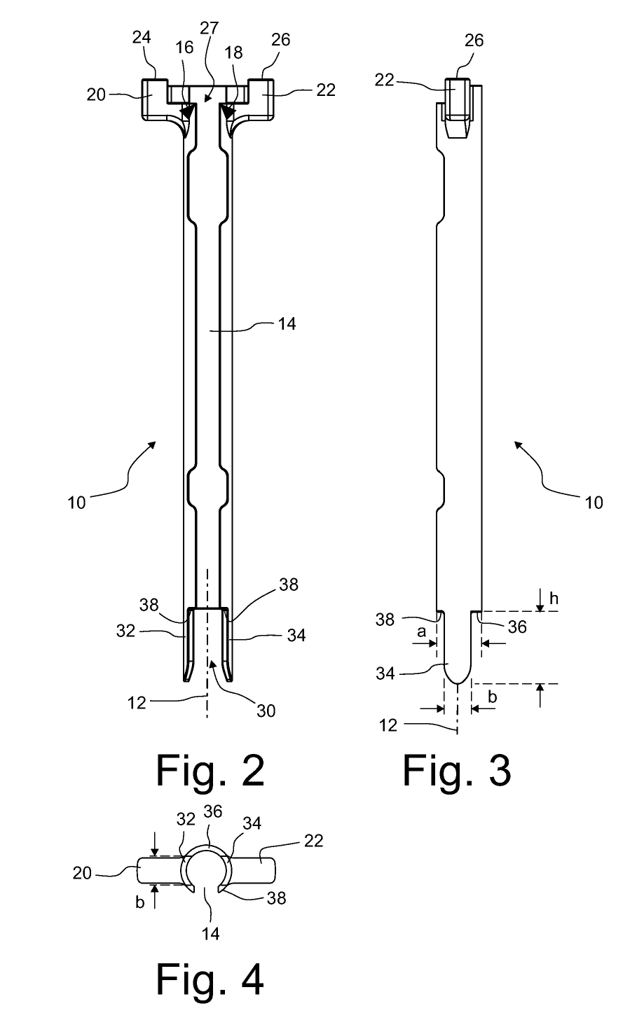

The guide sleeve 10 has substantially an annular cross-section; other cross-sections are also possible, however, e.g. square cross-sections. Extending parallelwise in relation to a longitudinal axis 12 of the guide sleeve 10 is a longitudinal groove 14, so extending continuously from the proximal end as far as the distal end of the guide sleeve 10. As can best be seen in FIG. 2, disposed at the proximal end of the guide sleeve 10 are two markings 16, 18, in the form of arrow tips, which can be printed onto the guide sleeve 10 or engraved into it. Realized in the region of the markings 18, 20 are two projections 20, 22, which are diametrically opposite one another and project radially from the longitudinal axis 12 of the guide sleeve 10. The proximal end surfaces of the two projections 20, 22 constitute action surfaces 24, 26, which are flat and run perpendicularly in relation to the longitudinal axis 12 of the guide sleeve 10. The action surfaces 24, 26 can be used to drive the distal end of the guide sleeve 10 into the intervertebral disc space by means of a hammer or another striking instrument. This is successful even when another instrument, e.g. a distractor or a screwing-in instrument previously introduced into the guide sleeve 10 protrudes from the proximal end of the guide sleeve 10. Extending between the projections 20, 22 is a web 27, which is somewhat lower in the axial direction than the projections 20, 22.

At its distal end, the guide sleeve 10 has two cut-outs 28, 30, which are diametrically opposite one another and between which there remain two spacer tongues 32, 34, which are opposite one another. The spacer tongues 32, 34 are thus constituted by a portion of the wall of the guide sleeve, but are rounded approximately elliptically towards the distal end, as can best be seen in the side view of FIG. 3.

The proximal end face of the cut-out 28 constitutes a first bearing shoulder 36 (see FIGS. 3 and 4), while the opposite cut-out 30, because of the longitudinal groove 14 extending there, defines two narrower, second bearing shoulders 38.

For explanation of the functionally important dimensions of the spacer tongues 32, 34, reference is made in the following to FIGS. 3 and 4. Along a transversal direction, which runs perpendicularly in relation to the longitudinal axis 12, the two spacer tongues 32, 34 have a width b, and along the longitudinal direction 12 they have a height h. The guide sleeves of the system according to the invention differ from one another in the widths b of the spacer tongues 32, 34, but also in the inside diameter and the outside diameter of the guide sleeves 10.

### b) Screwing-in Instrument

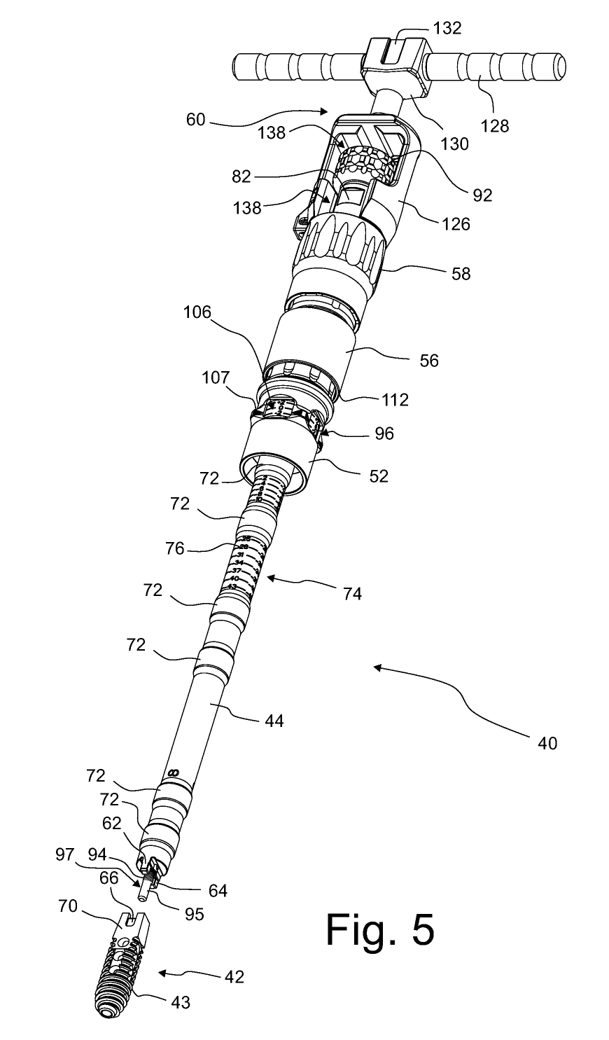

The structure of a screwing-in instrument according to the invention is explained in the following with reference to FIGS. 5 to 13, which show the screwing-in instrument, denoted as a whole by 40, in various views and sections. The description begins with an overview of the essential components of the screwing-in instrument 40. This is followed by a more detailed presentation of the structure and combined action of these components.

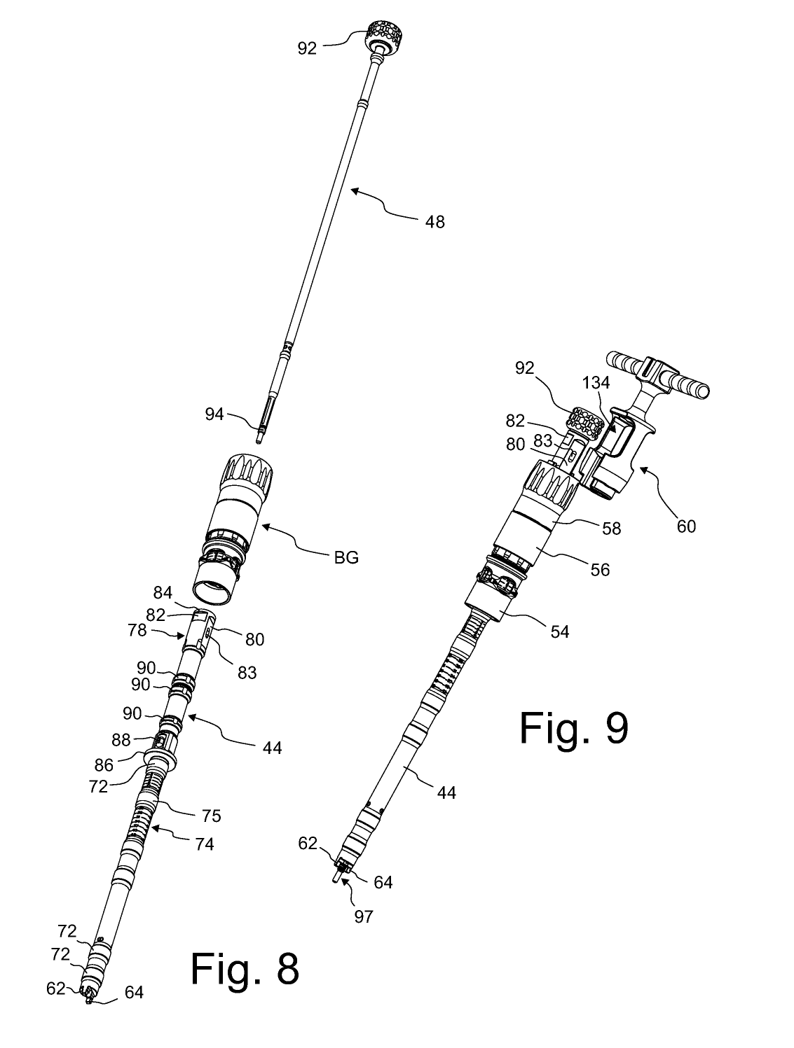

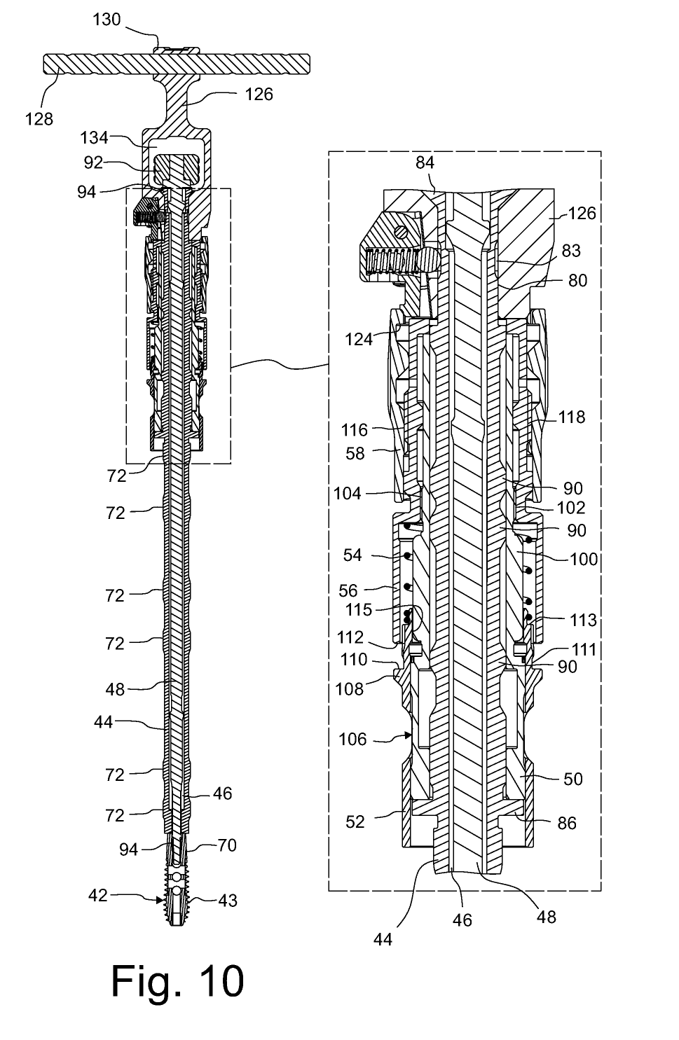

Reference is made first to FIG. 5, which shows a perspective representation of the fully assembled screwing-in instrument 40 and of an implant 42 to be screwed in, having an external thread 43. The screwing-in instrument 40 has a shank 44, which is provided with an axial through-bore 46, as best seen in the axial section of FIG. 10. Guided in the through-bore 46 of the shank 44 is a fastening rod 48, which is shown clearly in isolation in the exploded representation of FIG. 8.

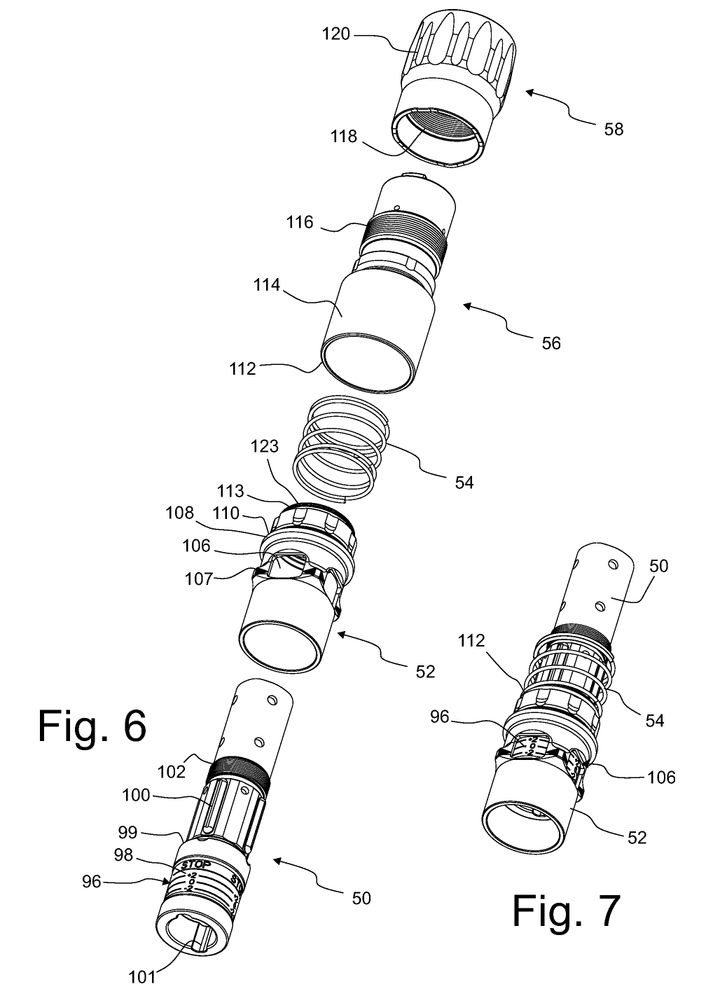

Pushed onto the shank 44 from its proximal end, which faces upwards in FIG. 8, is an inner sleeve 50, shown best in isolation at the bottom of FIG. 6, to which further reference is also made in the following. After being pushed on, the inner sleeve 50 is fixed to the shank 44 in a rotationally rigid manner. Guided in an axially movable manner on the inner sleeve 50 is an indictor sleeve 52, which, under the pressure of a compression spring 54, is supported on the inner sleeve 52. The compression spring 54, in turn, is fixed to a stop sleeve 56, which is screwed to the inner sleeve 50. Screwed onto the stop sleeve 56 is a latching sleeve 58, by means of which the inner sleeve 50, the indicator sleeve 52, the compression spring 54 and the stop sleeve 56 are tensioned relative to a handle unit 60, which can be seen, for instance, in FIGS. 5 and 9 and which, secured against axial displacement, is pushed laterally onto the shank 44.

The components of the screwing-in instrument 40 that have been described above, and their combined action, are described more fully in the following.

The shank 44, which can be seen best in its entirety in the exploded representation of FIG. 8, has two guide lugs 62, 64 at its distal end, which faces downwards in FIG. 8. As can best be seen in FIG. 5, the guide lugs 62, 64 have a shape that complements the geometry of guide grooves 66 realized in a base portion 70 of the implant 42. The latter has a rectangular cross-section, i.e. the dimensions differ in orthogonal directions that are perpendicular to the longitudinal axis.

Further realized on the shank 44 are a plurality of guide rings 72, in the regions of which the diameter of the shank 44 is enlarged. The diameter of the guide rings 72 in this case is slightly less than the inside diameter of the guide sleeve 10, such that the shank 44 can be introduced with a small clearance into the guide sleeve, and remains there in an axially movable manner.

A first scale portion 74, which is interrupted in a region 75 by one of the guide rings 72, is located, for instance, in the middle of the shank 44. It can be seen in FIG. 5 that the scale applied to the shank 44 there is provided with numbers 76, the meaning of which is explained later in connection with the functional description.

Located at the proximal end of the shank 44 there is a head portion 78, the shape of which can best be seen in FIG. 8. The head portion 78 is provided with a first parallelepiped 80 having larger plane surfaces and, turned by 90° relative thereto, is provided with a second parallelepiped 82 having smaller plane surfaces. Grooves 83 extending in the axial direction are in this case recessed-in on the larger plane surfaces of the first parallelepiped 80. At the distal end, the head portion 78 carries a cover ring 84, the diameter of which is less than the inside diameter of the inner sleeve 50, such that the latter can be pushed on over the proximal end of the shank 44.

Shown in FIG. 8, between the first scale portion 74 and the head portion 78, is support ring 86, which is realized on the shank 44 and whose diameter is greater than the inside diameter of the inner sleeve 50. Realized immediately adjacently on the shank 44 are short guide ribs 88, which extend in the axial direction and which, in a manner yet to be explained in greater detail, act together with the inner sleeve 50. Disposed between the guide ribs 88 and the head portion 78 are second guide rings 90, whose diameter is somewhat less than the inside diameter of the inner sleeve 50. The inner sleeve 50 can thereby be guided axially and pushed onto the shank 44 with sufficient clearance. In comparison with a shank 44 realized so as to be thicker overall, a saving in material and a reduction in weight are thereby achieved.

The fastening rod 48, which can best be seen in FIG. 8 and which, in the assembled state, extends through the through-bore 46 of the shank 44, is provided with a knurled locking handle 92 at its proximal end, and with an external thread 94 in the proximity of the distal end. The external thread 94 in this case is disposed at some distance from the distal end of the fastening rod 48, as shown clearly in FIG. 5. In the assembled state, the portion provided with the external thread 94 and the non-threaded portion 95 that adjoins the latter distally protrude out of the shank 44, and jointly constitute a stub 97.

In the following, unless otherwise stated, reference is made to FIG. 6. The inner sleeve 50 is provided with a second scale portion 96 in the proximity of its distal end, and inscribed with numbers 98. Towards the proximal end, the second scale portion 96 terminates with the inscription STOP.

Above the second scale portion 96, the inner sleeve 50 has a full-perimeter offset 99, adjoining which on the perimeter are four guide ribs 100 extending in the axial direction. The latter are followed by an external thread 102 that acts together with an internal thread 104, which is realized on the inside of the stop sleeve 56, and which can be seen only in the axial section of FIG. 10.

The inner sleeve 50 is provided on the inside with two longitudinal grooves 101 that are diametrically opposite one another.

The indicator sleeve 52 has four viewing windows 106 having an approximately rectangular contour. Between the viewing windows, markings 107 are applied to the indicator sleeve 52 from the outside in the manner of arrow tips. Further, the indicator sleeve 52 has a full-perimeter collar 108, which, on its proximal side, constitutes a shoulder 110 that is followed by an annular groove 111, which can be seen in FIG. 10. The shoulder 110 stops against an annular end face 112 of the stop sleeve 56 when the indictor sleeve 52 is displaced sufficiently far axially against the resistance of the compression spring 54. The compression spring 54 in this case is supported on an offset 113 realized at the proximal end of the indictor sleeve 52.

On the inside, the indicator sleeve 52, at its proximal end, is provided with four equidistant grooves, indicated at 115 in FIG. 10, which extend axially and which are realized to complement the guide ribs 100 on the inner sleeve 50. When the indicator sleeve 52 is pushed onto the inner sleeve 50, the grooves 115 and the guide ribs 100 ensure that the indicator sleeve 52 is fixed to the inner sleeve 50 in a rotationally rigid but axially movable manner.

The stop sleeve 56 has a distal portion 114, the inside diameter of which is dimensioned such that it can be pushed over the proximal end of the inner sleeve 50, the portion of the indicator sleeve 52 that remains proximally beyond the collar 108, and over the compression spring 54. The top sleeve 56 further carries an external thread 116, which can be screwed to an internal thread 118 realized on the latching sleeve 58.

On the outside, the latching sleeve 58 is provided with a knurling 120, to enable it to be grasped securely as it is being screwed onto the stop sleeve 56. At its proximal end, the latching sleeve 58 is provided, on the inside, with a plurality of equidistant grooves 122, which can best be seen in the cross-section of FIG. 13. Beneath the grooves 122, the wall of the latching sleeve 58 has a full-perimeter undercut 124, which can be seen in FIG. 10 and which, together with the grooves 122, provides for latching with the handle unit 60 in a manner yet to be explained in greater detail.

The handle unit 60, which can best be seen in the axial section of FIG. 11, comprises a handle housing 126, integrally realized on which there is a rotary handle 128. The connecting portion, indicated at 130, between the rotary handle 128 and the handle housing 126 is rectangular in cross-section, as can best be seen in FIG. 5. Realized on the outside of the connecting portion 130 are two strip-type grooves 132, which, as can also be seen in FIG. 5, are aligned in exactly the same way as the guide lugs 62, 64 at the distal end of the shank 44 when the screwing-in instrument 40 is in the assembled state.

As can best be seen in FIGS. 9 and 11, the handle housing 126 of the handle unit 60 has a large window 134, which serves to receive the locking handle 92 realized on the fastening rod 48, such that this locking handle can still also be actuated in the assembled state. Adjoining the window 134 is a packing 136, which is open on one side and whose opening 138, which can be seen, for instance, in FIGS. 5 and 13, is dimensioned in such a way that the handle unit 60 can be placed laterally onto the head portion 78 of the shank 44 (cf. figure). As shown by the cross-section of FIG. 13, plane-parallel inner surfaces 140, 142 of the packing 136 in this case bear positively on the plane surfaces of the first parallelepiped 80 of the head portion 78, whereby the handle unit 60 is fixed to the shank 44 in a rotationally rigid manner.

In addition, attached to the packing 136 of the handle housing 126 there is a swivel lever 146, which is provided with a latching hook 144 and which, together with the grooves 122 and the undercuts 124 of the latching sleeve 58, constitute a latching mechanism, such that the handle unit 60 is secured against being inadvertently drawn off laterally. The swivel lever 146, which can be swiveled about a swivel pin 148, is biased in this case by a small compression spring 150, which is supported on a ball 152. Owing to the pressure exerted by the compression spring 150, the ball 152, for its part, protrudes slightly from an opening 154 in the packing 136, but yields into the opening 154 upon pressure acting against the compression spring 150. When the handle unit 60 is placed onto the head portion 78 of the shank 44 as shown in FIG. 11, the ball 152 engages in one of the grooves 83 realized on the first parallelepiped 80 of the head portion 78. The ball 154, subjected to pressure and acting together with the grooves 83, thus constitutes an anti-loss device, which, when the screwing-in instrument 40 is assembled, ensures that the handle unit 60 pushed laterally onto the head portion 78 does not unintentionally become immediately detached again from the head portion 78 as soon as the handle unit 60 is released.

### II. Assembly of the Screwing-in Instrument

The assembly of the screwing-in instrument 40 is explained in the following.

In a first step, the indicator sleeve 52 is pushed onto the inner sleeve 50, the guide ribs 100 on the inner sleeve 50 engaging in the corresponding grooves realized on the inside of the indicator sleeve 52. In order to find one of the four possible angular relative positions, the indicator sleeve 52 in this case might have to be turned a little. An annular offset, not visible in the figures, on the inner surface of the indicator sleeve 52 stops in this case against the offset 99 that is realized on the inner sleeve 50. The compression spring 54 is then guided over the proximal end of the inner sleeve 50 until it sits on the offset 113 of the indicator sleeve 52 and latches in slightly. This assembly state is shown in FIG. 7.

In a following step, the stop sleeve 56, with the distal portion 114 foremost, is placed onto the assembly shown in FIG. 7 and screwed to the latter, the internal thread 104 of the stop sleeve 56 coming into engagement with the external thread 102 of the inner sleeve 50, and the compression spring 54 stopping against the stop sleeve 56, on the inside. The inner sleeve 50 and the stop sleeve 56 are now fixedly connected to one another, while the indicator sleeve 52 remains axially movable because of the guidance on the guide ribs 100. The indicator sleeve 52 can thus be displaced, against the resistance of the compression spring 54, to the distal end face 112 of the stop sleeve 56. During such an axial displacement, the viewing windows 106 realized on the indicator sleeve 52 sweep over the second scale portion 96 applied on the inner sleeve 50. At the same time, the markings 107 on the indicator sleeve 52 that are applied between the viewing windows 106 point to the scale and the numbers 98.

In a further step, the latching sleeve 58 is screwed onto the stop sleeve 56, the internal thread 118 of the latching sleeve 58 coming into engagement with the external thread 116 of the stop sleeve 56. The assembly obtained thus is represented in the middle of FIG. 8, beneath the fastening rod 48.

The assembly BG is now pushed on over the proximal end of the shank 44 until the distal end of the inner sleeve 50 stops against the support ring 86 of the shank 44. The guide ribs 88 above the support ring 86, together with the longitudinal grooves 101 provided on the inner surface of the inner sleeve 50, then ensure that the inner sleeve 50, and therefore the entire assembly shown in the middle of FIG. 8, is fixed to the shank 44 in a rotationally rigid manner.

In a further step, the fastening rod 48 is introduced, from the proximal end of the shank 44, into the through-bore 46 thereof, until an offset realized directly beneath the locking handle 92 of the fastening rod 48 stops against the cover ring 84 of the head portion 78 realized on the shank (cf. FIG. 10).

In a further step, the handle unit 60 is placed laterally onto the head portion 78, as shown in FIG. 9. When the handle unit 60 is in the end position, the ball 152, under compression-spring load, engages in one of the grooves 83 realized on the head portion 78, and thereby creates an anti-loss device.

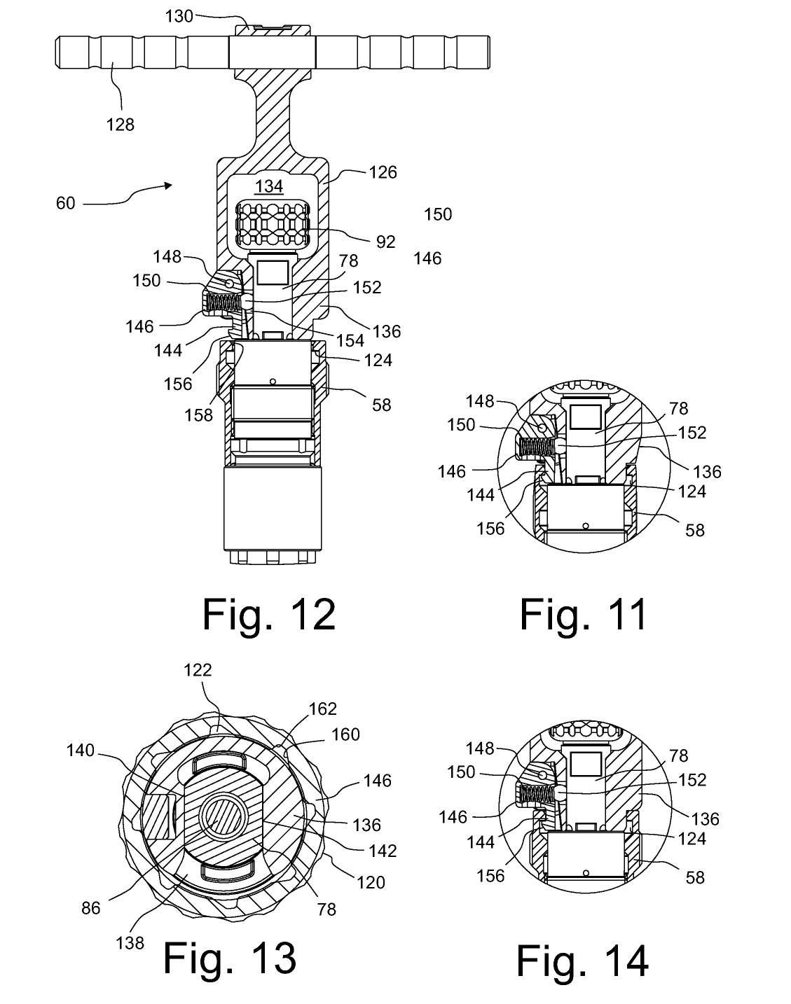

In a final step, the latching sleeve 58 is then released again and turned to move it in the direction of the handle unit 60. When the latching sleeve 58 is in the axial position shown in FIG. 11, the latching hook 144 does not yet bear against the locking sleeve 58. If, however, the latter is released further and thereby displaced towards the handle unit 60, an obliquely disposed slide surface 156 on the latching hook 144 acts on counter-surfaces 158, likewise running obliquely, at the distal end of the latching sleeve 58. Upon continued axial movement of the latching sleeve 58, the sliding of the slide surface 156 on the counter-surface 158 causes the swivel lever 146 to be swiveled about the swivel pin 148, as a result of which the latching hook 144 is moved radially inwards. Upon rotation of the latching sleeve 58 being continued further, the latching hook 144 finally snaps in behind one of the undercuts 124, as shown in the detail of FIG. 12. The compression spring 150 acting on the swivel lever 146 in this case prevents this axial latching from being released again.

At the same time, latching is also achieved in the direction of rotation, since the latching hook 144, when in certain rotary positions, can at the same time engage in one of the grooves 122 realized on the inside of the latching sleeve 58. In this case, sloping side surfaces 160 of the grooves 122, which can be seen in FIG. 13, act together with a likewise sloping side surface on the latching hook 144, as a result of which the latching sleeve 58 can only be turned in one direction of rotation, while, in the other direction of rotation, the latching hook 144 catches on radially extending latching surfaces 162 of the grooves 122. The direction of rotation in which the latching sleeve 58 can be rotated in this case is the direction in which the latching sleeve 58 can be brought closer to the handle unit 60.

Because of this latching mechanism, therefore, after the axial latching has occurred, as shown in FIG. 12, the latching sleeve 58 can no longer be turned back without releasing the latching again by manual pressure on the swivel lever 146. Further, the combined action of the latching hook 144 and the grooves 122 produces a recurrent click sound when the latching sleeve 58 is turned towards the handle unit 60. This click sound is also an acoustic indication that complete latching will happen shortly. The latching mechanism in this case can be designed, for example, such that, for instance, 3 to 6 sounds occur until complete latching has happened.

FIG. 14 shows, in a detail based on FIG. 12, an alternative configuration of a latching mechanism. In the case of this latching mechanism, the latching sleeve 58 is not provided with grooves 122, but contributes only a single full-perimeter undercut 124 to the latching mechanism. In the case of this configuration, the latching sleeve 58 therefore remains still rotationally movable in the axially latched state also, and no click sounds occur as the latching is approached.

Clearly, two latching mechanisms of the same type can also be provided. In particular, if these latching mechanisms are disposed diametrically opposite one another, tilting is thereby prevented in an effective manner.

### III. Use

The following describes, with reference to FIGS. 16 to 22, how the implant 42 is screwed into an intervertebral disc space by means of the instrument set described above.

In a first step, an access to the intervertebral disc space is exposed. This can be, for example, a posterior-lateral access. In most cases, the intervertebral disc is wholly or partially removed. In this case, the vertebrae delimiting the intervertebral disc space generally move towards one another, as a result of which the height of the intervertebral disc space is at first reduced.

In order to distract the intervertebral disc space again, a set of a plurality of distractors can be used, as described in the applicant's unpublished German patent application, “System for distracting an intervertebral disc space”, already mentioned above. The intervertebral disc space in this case is distracted successively through use of increasingly larger distractors, which each have a head that is elliptical in cross-section. The respectively obtained distraction is maintained in each step by means of a fitting guide sleeve 10. At the end of this procedure, the last distractor is withdrawn, such that the distraction is maintained solely by one of the guide sleeves 10. The size of the last guide sleeve 10 used depends in this case on, inter alia, the size of the intervertebral disc space and the dimensions of the implant 42 that is to be introduced into the intervertebral disc space.

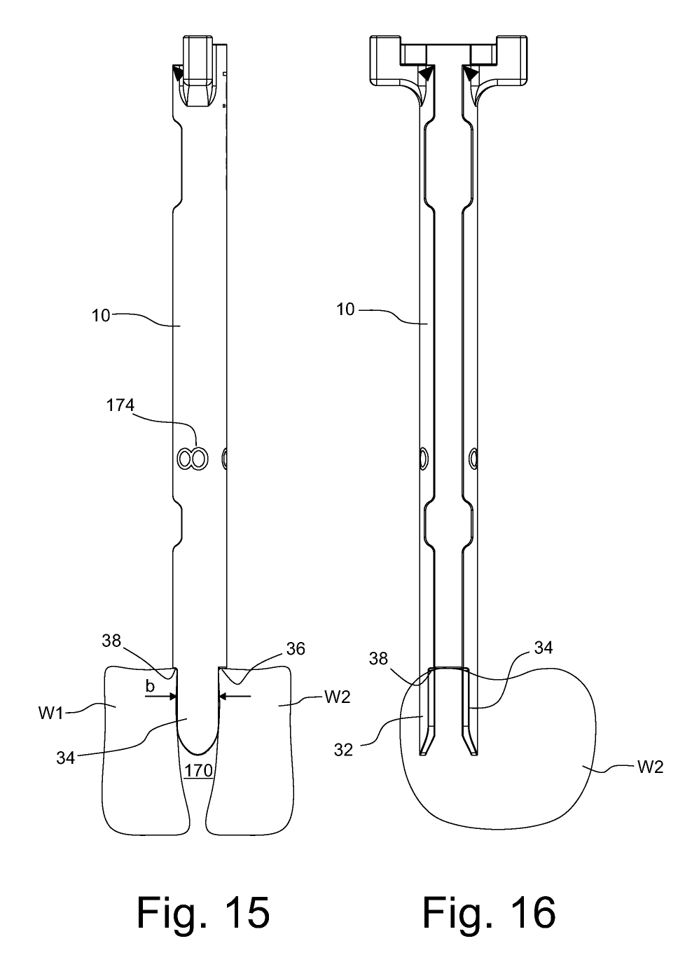

In FIGS. 15 and 16, this state is shown in a side view and a front view, respectively, of the guide sleeve 10. In the side view of FIG. 15, it can be seen that the two spacer tongues 32, 34 project into the intervertebral disc space, denoted by 170. The distraction of the intervertebral disc space 170 in this case is equal to the width b of the spacer tongues 32, 34. The shoulders 36, 38 realized at the distal end of the guide sleeve 10 are in this case supported on the edges of the vertebrae W1, W2, which delimit the intervertebral disc space 170.

In a second step, a screwing-in instrument 40 is prepared, which is matched to the implant 42 to be used and to the guide sleeve 10 selected for the latter. As already mentioned at the outset, the shank 44 is specially matched, with the guide lugs 62, 64, to the geometries of different implants 42. This also applies correspondingly to the fastening rod 48, whose diameter, in the case of the embodiment represented, is matched to an inner bore in the implant 42.

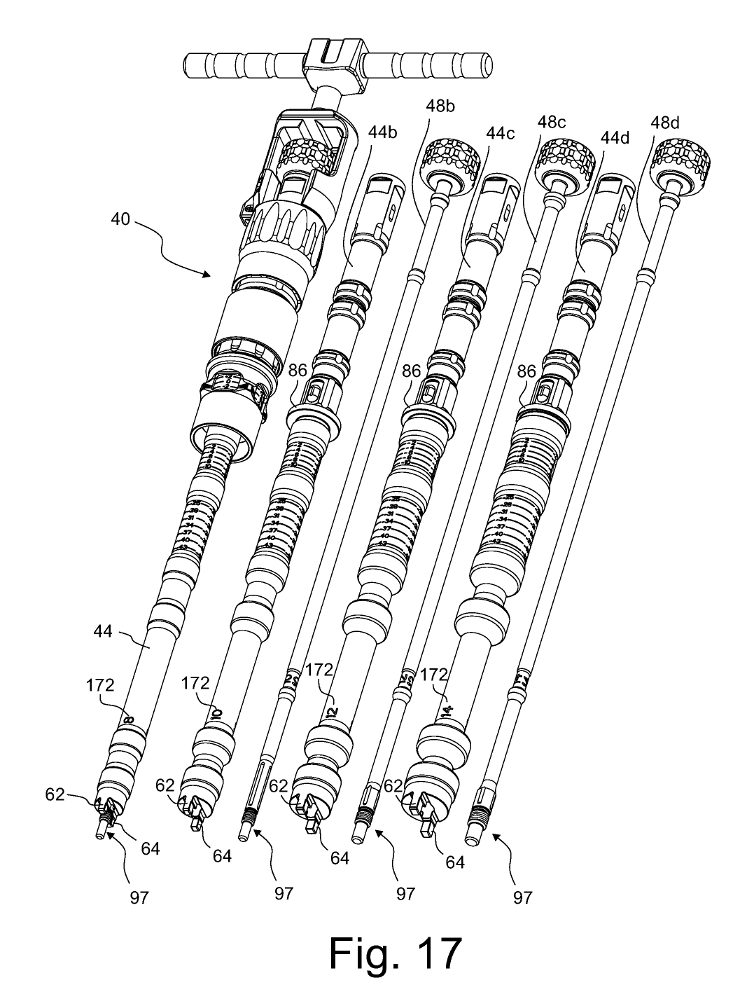

FIG. 17 shows exemplarily, in addition to a completely assembled screwing-in instrument 40 having a shank 44, further shanks 44b, 44c, 44d, which have different diameters between the support ring 86 and the distal end. These diameters and, in particular, the diameters of the first guide rings 72 are matched to guide sleeves 10 that differ in their inside diameter. In addition, the size of the guide lugs 62, 64 varies between some of the shanks 44, 44b, 44c, 44d.

Represented next to each of the shanks 44b, 44c, 44d there is a fastening rod 48b, 48c and 48d, respectively, which are matched thereto and which differ from one another in the diameter of the stub 97 and of the external thread 94 carried by the latter, and which can be introduced into only one of the shanks 44, 44b, 44c, 44d in each case.

In the embodiment shown, of the shanks 44, 44b, 44c, 44d provided with numbers 172, there has already been selected that shank 44 which matches the implant 42 and for which the fitting guide sleeve 10, represented in FIGS. 15 and 15 and provided with a corresponding number 174, has been chosen. If the implant 42 to be used requires a different shank 44b, 44c, 44d, the screwing-in instrument 40 shown on the left in FIG. 17 must be disassembled. For this purpose, the latching of the latching sleeve 58 is first released by actuating the swivel lever 146, the latching sleeve 58 is rotated downwards again, and the handle unit 60 is drawn off laterally. The fastening rod 48 can then be drawn out of the through-bore 46, and the assembly shown in the middle in FIG. 8 can be drawn off the shank 44. Such a conversion can be performed very rapidly, and even still during the operation. The assembly is then pushed onto one of the shanks 44b, 44c or 44d that are of different dimensions, and the assembly is continued in the manner described under II above.

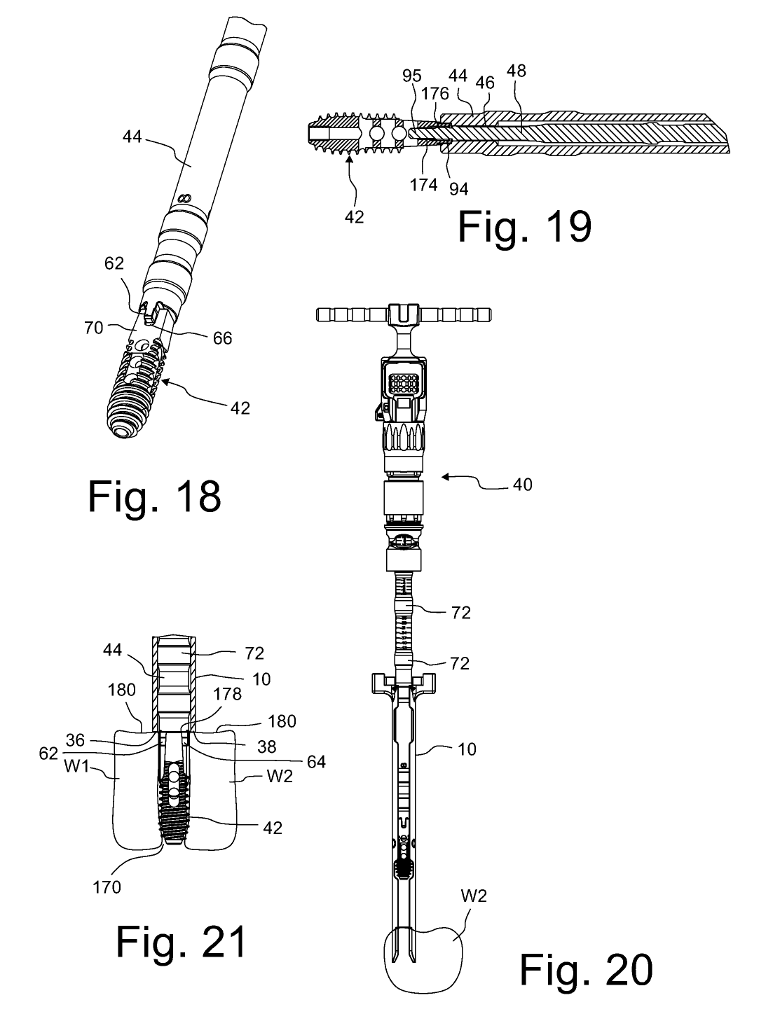

After the fitting screwing-in instrument 40 has been provided, the implant 42 is fastened to the distal end of the screwing-in instrument 40, in a third step. For this purpose, the implant 42 is placed onto the distal end of the shank 44, as indicated in FIG. 5, such that the guide lugs 62, 64 at the distal end of the shank 44 engage in the guide grooves 66 realized on the base portion 70 of the implant 42. In this state, shown by the perspective view of FIG. 18, the implant 42 is fixed on the shank 44 in a rotationally rigid manner and with a defined angular orientation.

As the implant 42 is placed on, the stub 97 of the fastening rod 48 that projects out of the shank 44 engages in an axial bore 174 of the implant 42, which bore can be seen in the axial section of FIG. 19. An upper portion of the axial bore 174 that faces towards the screwing-in instrument 40 is provided with an internal thread 176, which is matched to the recessed external thread 94 on the stub 97. The stub 97 and the arrangements of the threads 94, 176 are fixed in this case such that the external thread 94 on the stub 97 can only engage in the internal thread 176 of the implant 42 when the implant 42 has been fixed with the correct angular orientation on the shank 44, as shown in FIG. 18. In the case of an angular orientation turned by 90°, although it would be possible for the non-threaded, front portion of the stub 97 to be introduced a little further into the axial bore 174 of the implant 42, engagement of the threads 94, 176 would nevertheless not be possible. Consequently, the implant 42 could not be fastened to the screwing-in instrument 40 when in this incorrect angular orientation.

However, when the implant 42 is in the correct angular orientation in relation to the screwing-in instrument 40, as shown in FIG. 18, the threads 94, 176 come into engagement, such that the fastening rod 48 can be screwed into the implant 42 by turning the locking handle 92. The implant 42 thereby becomes clamped to the shank 44 and is now also secured axially.

In a fourth step, the screwing-in instrument 40, with the implant 42 fastened thereto, is introduced into the guide sleeve 10, as shown in the front view of FIG. 20. The first guide rings 72 in this case ensure a coaxial alignment of the shank 44 and guide sleeve 10, whereby tilting is prevented. Before the introduction procedure, a bore can also be produced in the intervertebral disc space 170 by means of a drill, and this also possibly over only a part of the entire length of the implant 42.

FIG. 21 shows, in a detail, the subsequent specified position of the implant 42 in the intervertebral disc space 170; the front part of the sleeve 10 has been removed in this case to show the shank 44 of the screwing-in instrument 40 guided therein. This specified position is characterized in that the proximal end surface of the implant 42, which is denoted by 178, is flush with the rear edges 180 of the vertebrae W1, W2. In this specified position, the proximal end surface 178 is therefore at the same level as the shoulders 36, 38 of the guide sleeve 10, which are supported on the vertebrae W1, W2.

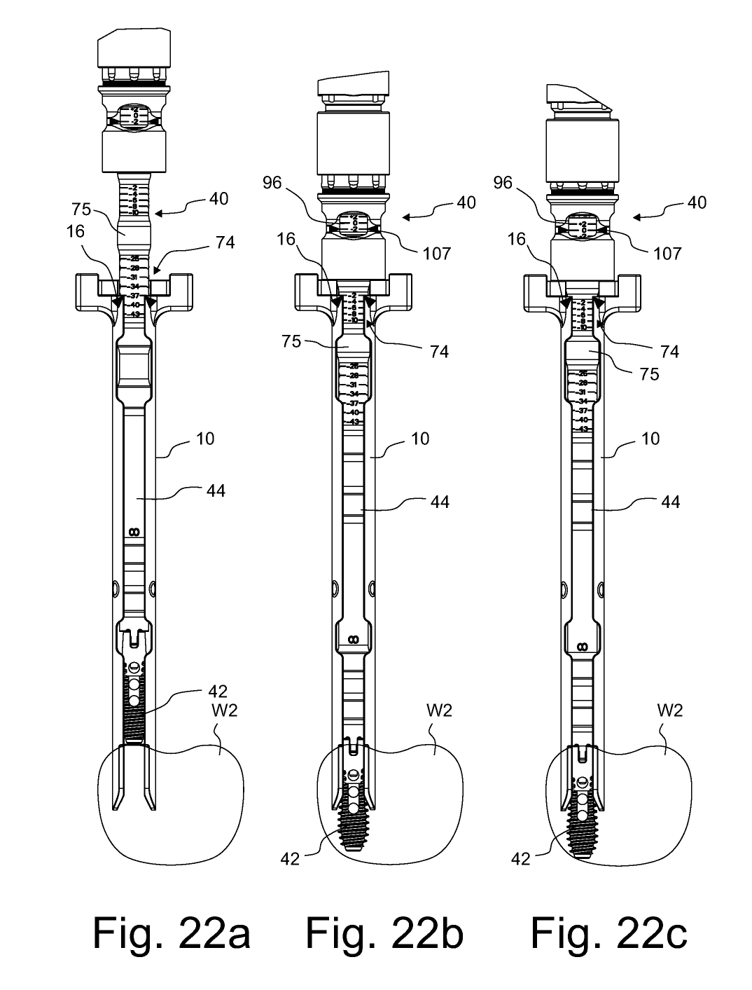

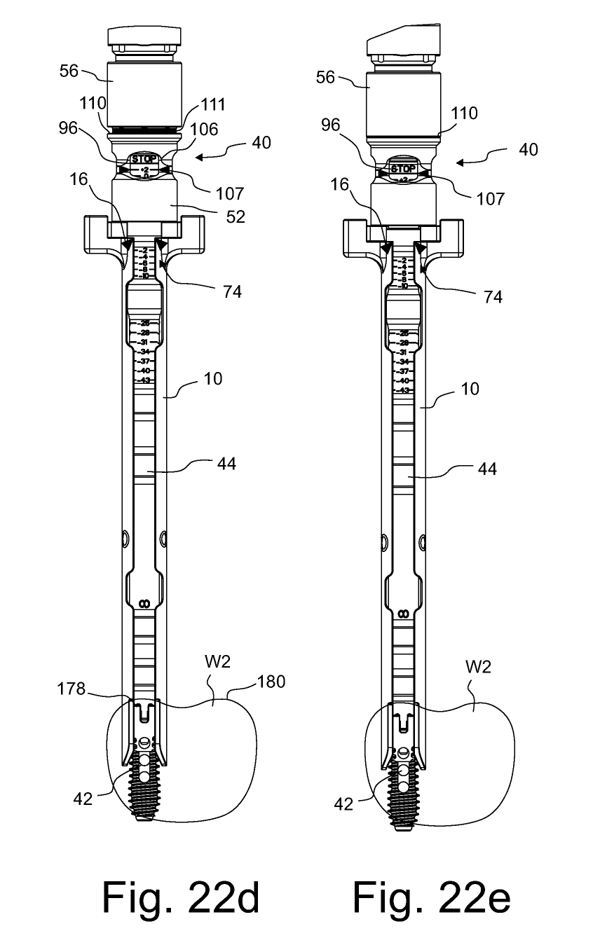

As soon as the distal end of the implant 42 stops against the edges 180 of the vertebrae W1, W2, as shown in FIG. 22a, screwing of the implant 42 into the intervertebral disc space 170 can commence, in a fifth step. In the case of the starting position shown in FIG. 22a, the tips of the marking 16, provided on the guide sleeve 10, on the first scale portion 74 of the shank 44 point to a number value that indicates the length of the implant 42. In this way, the surgeon can once again make certain that the implant 42, fastened to the distal end of the shank 44 and now no longer visible, has the desired length. Because of the fixing of the specified position of the implant 42, explained with reference to FIG. 21, the value indicated on the first scale portion 74 corresponds at the same time to the residual advance still required to move the implant 42 to its specified position shown in FIG. 21.

For the purpose of driving in the implant 42, the rotary handle 128 on the handle unit 60 is now rotated clockwise. By means of the parallelepiped 80 on the shank 44, the rotation of the handle unit 60 is transferred to the shank 44 and, via the guide lugs 62, 64 realized there, to the implant 42. The latter now screws into the intervertebral disc space, the implant 42 preferably turning its thread itself.

As the implant 42 is screwed into the intervertebral disc space 70, the entire screwing-in instrument 40 is advanced continuously in the axial direction. Throughout this, the distance of the implant 42 from its axial specified position can be read off from the first scale portion 74. In the case of the embodiment represented, the first scale portion is interrupted in the region 75, since the required residual advance is still relatively large, and continuous reading-off of the residual advance would possibly only distract the surgeon. The first scale portion 74 is continued again only when only just −10 mm of residual advance remain.

As soon as only just −2 mm of residual advance remain, the distal edge of the indicator sleeve 52 stops against the web 27 that extends between the projections 20, 22 of the guide sleeve 10. This state is shown in FIG. 22b. The second scale portion 96 on the inner sleeve 50 in this case is matched to the first scale portion 74 such that, in this axial position, the residual advance of −2 mm that can be read off from the first scale portion 74 can also be read off, through the viewing window 106, from the second scale portion 96, at the level of the markings 107 of the indicator sleeve 52. The stopping of the indicator sleeve 52 against the guide sleeve 10 and the axial displacement of the indicator sleeve 52 that then ensues indicate to the surgeon, in an optical manner that is clearly discernible, that the implant 42 is now approaching its specified position and that the screwing-in procedure must now be continued with particular care, in order that the implant 42 is not inadvertently screwed too far into the intervertebral disc space 170.

FIG. 22c shows the arrangement of the screwing-in instrument 40 and of the guide sleeve 10 at the point in time at which the implant 42 has reached its specified position shown in FIG. 21. For the surgeon, this can be identified in that the residual advance is exactly 0 mm in the viewing window 106 of the indicator sleeve 52. The surgeon can now additionally use imaging methods to check that the correct specified position has been achieved.

If the surgeon concludes that the implant 42 is optimally positioned in the intervertebral disc space 170, he can still fix the desired angular orientation of the implant 42 in the intervertebral disc space 170. Since the implant 42 has a rectangular cross-section in the region of the base portion 70, one and the same implant 42 can fix two different angles between the vertebrae W1, W2, namely, depending on the angular orientation in which the implant 42 is located in the intervertebral disc space 170. The surgeon identifies the angular orientation of the implant 42 from the connecting portion 130 of the handle unit 60, which, just like the base portion 70 of the implant 42, has a rectangular cross-section. In order to change back and forth between the two possible angular orientations of the vertebrae W1, W2, the surgeon only has to move the rotary handle 128 of the handle unit 60 back and forth between two angular orientations turned by 90° relative to one another. Owing to the relatively small pitch of the external thread 43 of the implant 42, its axial position in the intervertebral disc space 170 scarcely alters as a result.

Once the angular orientation also has been fixed in the desired manner, the surgeon, in a sixth step, releases the fastening of the implant 42 to the shank 44 in that the surgeon, by actuating the locking handle 92, releases the screwed connection between the fastening rod 48 and the implant 42. The screwing-in instrument 40 can now be drawn out of the guide sleeve 10, while the implant 42 remains in its specified position in the intervertebral disc space 170.

If, in the checking by means of imaging methods, the surgeon determines that slight corrections are still required, he can still perform these corrections before the connection to the implant 42 is released. The second scale portion 96 in the viewing window 106 of the indicator sleeve 52 continuously indicates to the surgeon in this case how far distant he is from the specified position (i.e. residual advance=0 mm) in both directions.

Shown exemplarily in FIG. 22d is a state in which the surgeon has screwed the implant 42 into the intervertebral disc space 170 to just past the specified position. In this position, the proximal end surface 178 of the implant 42 is no longer located at the level of the rear edges 180 of the vertebrae W1, W2, as can be seen in FIG. 22d. For this reason, in the viewing window 106 of the indicator sleeve 52, the second scale portion 77 indicates, by a value of +2 mm, that the implant is already located 2 mm past its specified position in the intervertebral disc space 170. Further, in the case of the implant 42 being in this axial position, the imprint STOP appears in the viewing window 106 of the indicator sleeve 52, whereby it is indicated to the surgeon that the screwing-in procedure must now not be continued further, in order to avoid damage to sensitive tissue by the implant 42. In this position, the annular groove 111 realized above the offset 113 on the indicator sleeve 52 is located directly beneath the stop sleeve 56, whereby a kind of change in brightness is achieved that additionally draws the surgeon's attention to the fact that the axial specified position of the implant 42 has already been exceeded and that the indicator sleeve 52 is shortly about to stop against the stop sleeve 58.

If the surgeon nevertheless continues the screwing-in procedure, the shoulder 110 of the indicator sleeve 52 finally stops against the stop sleeve 56, such that the shank 44, fixed axially relative to the stop sleeve 56, cannot be lowered further. At the same time, the inscription STOP appears between the markings 107 in the viewing window 106 of the indicator sleeve 52. This state is shown in FIG. 22e.

This limitation of the axial penetration depth, effected by the stop, is achieved without the surgeon having first to adjust and fix an adjustable stop on the shank 44. Since such adjustment procedures are subject to error, the surgeon cannot rely to an unlimited extent on such an adjustable stop being set correctly. In the case of the screwing-in instrument 40 according to the invention, on the other hand, no such adjustable stops are required. Consequently, even without any checking by imaging methods and the radiation load associated with the same, the surgeon could safely screw the implant 42 into its specified position and still adjust it axially there, within certain limits. Through the combined action of the guide sleeves 10 and the screwing-in instrument 40 specially matched thereto, the instrument set according to the invention achieves the reliable stop that delimits the penetration depth.

1. An instrument set for screwing an implant provided with an external thread into an intervertebral disc space, wherein the instrument set comprises:
a guide sleeve, which
has a distal end which rests, during the screwing-in procedure, on vertebrae delimiting the intervertebral disc space, and
has a proximal end, which faces towards the surgeon during the screwing-in procedure;
a screwing-in instrument, which has a shank that is configured such that it can be introduced into the guide sleeve, wherein the shank has
a distal end, to which the implant can be fixed in a rotationally rigid manner, and
a proximal end, to which a rotary handle is fastened in a rotationally rigid manner and which projects over the proximal end of the guide sleeve during the screwing-in procedure,
wherein the screwing-in instrument further has a residual-advance indicator, which is configured to quantitatively indicate to the surgeon, during the screwing-in procedure, how far the implant is distant in the axial direction from an axial specified position that is fixed relative to the location at which the guide sleeve is supported on the vertebrae.

2. The instrument set of claim 1, wherein the residual-advance indicator is configured such that it also quantitatively indicates exceeding of the specified position in the axial direction.

3. The instrument set of claim 1, wherein the residual-advance indicator comprises a scale, which is applied to the shank or to a component fixed axially thereto, and which acts together with the guide sleeve.

4. The instrument set of claim 3, wherein the proximal end of the guide sleeve is provided with a marking, which is directed towards the scale when the screwing-in instrument has been introduced into the guide sleeve.

5. The instrument set of claim 3, wherein the screwing-in instrument has an indicator sleeve, which is guided so as to be axially movable relative to the shank and which, during the introduction of the screwing-in instrument into the guide sleeve, or during the screwing-in procedure, stops against the proximal end of the guide sleeve, and is provided with a viewing window, through which the scale can be seen.

6. The instrument set of claim 5, wherein the edge of the viewing window is provided with a marking directed towards the scale.

7. The instrument set of claim 5, wherein the screwing-in instrument has an elastic element, which biases the indicator sleeve in the axial direction in such a way that, during the screwing-in procedure, the indicator sleeve is displaced axially relative to the shank against the action of the elastic element.

8. The instrument set of claim 5, wherein the scale has a first scale portion, which acts together with the marking on the guide sleeve, and a second scale portion, which acts together with the marking on the indicator sleeve.

9. The instrument set of claim 8, wherein the two scale portions are matched to one another in such a way that, when the indicator sleeve stops against the proximal end of the guide sleeve during the screwing-in procedure, it is solely the second scale portion that quantitatively indicates the residual advance and, possibly, exceeding of the specified position in the axial direction.

10. The instrument set of claim 1, wherein the shank of the screwing-in instrument has a through-bore extending over the entire length of the shank, and wherein the screwing-in instrument has a fastening rod, which is guided in the through-bore and which, at its distal end, is provided with a fastening portion by means of which the implant can be fixed to the fastening rod in the axial direction.

11. The instrument set of claim 1, wherein the rotary handle is detachably fastened to the shank.

12. The instrument set of claim 11, wherein, for the purpose of fastening the rotary handle to the shank, the screwing-in instrument has a latching mechanism, and wherein a latching of the rotary handle with the shank is releasable only through actuation of a spring-loaded latching button.

13. The instrument of claim 12, wherein the fixing of the rotary handle to the shank in a rotationally rigid manner is achieved by positive locking, and wherein the latching mechanism fixes the rotary handle in the axial direction relative to the shank.

14. The instrument set of claim 13, wherein the latching mechanism has a latching sleeve, which is screwed onto the shank and which, when in the latched state, is fixed in position axially and in a rotationally rigid manner on the shank.

15. An implantation system comprising:
an implant that is provided with an external thread and is configured for being screwed into an intervertebral disc space,
an instrument set according to claim 1,
wherein the implant has different dimensions in two orthogonal directions that run perpendicularly in relation to a longitudinal axis of the implant in such a way that, depending on the angular orientation of the implant in the intervertebral disc space, different distances and/or angles between the vertebrae delimiting the intervertebral disc space are obtained, and wherein a fastening portion of the screwing-in instrument is configured in such a way that the implant can be fixed to the screwing-in instrument in a rotationally rigid manner only in precisely two angular orientations, which differ from one another by 180°.

16. The implantation system of claim 15, wherein the rotary handle on the screwing-in instrument has a portion that, in a section perpendicular to the longitudinal axis of the shank, has at least substantially the shape of a rectangle, wherein the long sides of the rectangle run parallel to the direction along which the dimensions of the implant fixed to the screwing-in instrument are greater.

17. The implantation system of claim 15, wherein the fastening portion comprises an external thread that is disposed axially in such a way that only in the two angular orientations can it engage in an internal thread on the implant.

18. The implantation system of claim 15, wherein the system further comprises
a plurality of shanks, which differ from one another in the design of the fastening portions,
a plurality of implants of different sizes, which have fastening means by which they can each be fixed in a rotationally rigid manner to the fastening portion of one of the shanks, wherein at least two implants that differ in size also have differently designed fastening means, and
a plurality of guide sleeves having inside diameters that are matched to the diameters of the implants and of the shanks
